SEQUENCE LISTING (8 sequences)

SEQ ID NO: 1 - protein, 135 aa, Agaricus bisporus
MFSKVYLVASTLIAVAVAQAPLQCYQGLPTSAGPATDCSRFVNTFCDAAAAVPAVRINDSVSRCFNLPDAKVCDFIAWNTFTRNVPPSAANCKSVLNKVISQCVLGGYGQVGPNAYTFTVDVNHGQCGHDVHGGS

SEQ ID NO: 2 - DNA, 408 bp, Agaricus bisporus
atgttttccaaagtctatcttgttgcatctactctcatcgctgttgcggtggctcaagcacctctccagtgctaccaaggcttacccacttctgcgggtcccgcaactgactgttcccgatttgtgaacaccttttgcgacgcggctgccgctgtccctgcggtacgcattaacgattcagtcagtcgttgcttcaatttaccagatgccaaagtttgcgatttcattgcgtggaacacattcacgcgcaacgttccgccttcagcggctaattgcaaatccgtgctgaacaaagttatttctcaatgcgtattgggaggttatggacaggttgggcccaatgcgtatactttcacggttgacgtcaaccatggacagtgtggccatgatgtccatggtggatcctag

SEQ ID NO: 3 - protein, 117 aa, Agaricus bisporus
QAPLQCYQGLPTSAGPATDCSRFVNTFCDAAAAVPAVRINDSVSRCFNLPDAKVCDFIAWNTFTRNVPPSAANCKSVLNKVISQCVLGGYGQVGPNAYTFTVDVNHGQCGHDVHGGS

SEQ ID NO: 4 - DNA, 351 bp, Agaricus bisporus
caggctccattgcagtgctatcagggtttgccaacctctgctggtccggccaccgattgctcccgcttcgtgaataccttctgtgacgcggctgcagcggttccggcagtgcgtattaatgatagcgttagccgttgctttaacctgccggatgcgaaagtgtgtgactttatcgcttggaataccttcacccgtaacgttccgccgagcgctgcaaattgcaaaagcgttctgaacaaagttattagccaatgtgttctgggtggttacggtcaagtgggtccgaatgcttacaccttcaccgtggatgttaatcatggtcaatgtggtcacgatgttcatggcggctcc

SEQ ID NO: 5 - DNA, 18 bp, Agaricus bisporus
gtattcaggatagcgtta

SEQ ID NO: 6 - DNA, 18 bp, Agaricus bisporus
ctatcctgaatacgcact

SEQ ID NO: 7 - protein, 112 aa, Agaricus bisporus
QDPLSCYDNFGNRDVAACARFIDDFCDTLTPNIYRPRDNGQRCYVVNGHKCDFTVFNTNNGGSPIRASTPNCKTVLRAAANRCPTGGRGKINPSAPFLFAIDPNDGDCSTDF

SEQ ID NO: 8 - DNA, 354 bp, Agaricus bisporus
caggctccattgcagtgctatcagggtttgccaacctctgctggtccggccaccgattgctcccgcttcgtgaataccttctgtgacgcggctgcagcggttccggcagtgcgtattaatgatagcgttagccgttgctttaacctgccggatgcgaaagtgtgtgactttatcgcttggaataccttcacccgtaacgttccgccgagcgctgcaaattgcaaaagcgttctgaacaaagttattagccaatgtgttctgggtggttacggtcaagtgggtccgaatgcttacaccttcaccgtggatgttaatcatggtcaatgtggtcacgatgttcatggcggctcctaa

(19) United States
(12) Patent Application Publication
(10) Pub. No.: US 2022/0227820 A1
(43) Pub. Date: Jul. 21, 2022

(54) RECOMBINANT GLYCAN BINDING PROTEINS

(71) Applicant: University of Florida Research Foundation, Incorporated, Gainesville, FL (US)

(72) Inventors: Yousong Ding, Gainesville, FL (US); Steven Douglas Bruner, Gainesville, FL (US); Peilan Zhang, Gainesville, FL (US); Brian MacTavish, Gainesville, FL (US)

(73) Assignee: University of Florida Research Foundation, Incorporated, Gainesville, FL (US)

(21) Appl. No.: 17/605,616

(22) PCT Filed: Apr. 23, 2020
(86) PCT No.: PCT/US2020/029643

§ 371 Date: Oct. 22, 2021

Related U.S. Application Data
(60) Provisional application No. 62/838,408, filed on Apr. 25, 2019.

(51) Int. Cl. C07K 14/375; C12N 15/81; G01N 33/68; C07K 1/14

(52) U.S. Cl. CPC C07K 14/375; C12N 15/815; G01N 33/68; G01N 2333/4728; G01N 2400/02; G01N 2333/375; C07K 1/145

(57) ABSTRACT

Aspects of the disclosure relate to compositions and methods for expressing glycan binding proteins (GBPs) in a cell or cells. In some embodiments, the disclosure provides recombinant Ab-Y3 protein variants and isolated nucleic acids engineered to express the same. In some embodiments, the disclosure provides methods of isolating, purifying, detecting, or screening for certain proteins (e.g., IgG proteins) using recombinant Ab-Y3 protein variants.

### RELATED APPLICATIONS

This Application is a national stage filing under 35 U.S.C. 371 of International Patent Application Serial No. PCT/US2020/029643, filed Apr. 23, 2020, which claims the benefit under 35 U.S.C. 119(e) of U.S. Provisional Patent Application Ser. No. 62/838,408, filed Apr. 25, 2019. The entire contents of these applications are incorporated herein by reference in their entirety.

### BACKGROUND

Glycans are one fundamental building block to all life forms in nature, which have long been known to play major physical, metabolic and structural roles in biological systems. The recognition of remarkably complex glycans is often mediated by the complementary glycan binding proteins (GBPs) that carry specific glycan-recognition domains. As such, GBPs read and execute biological information encoded in diverse glycans, being essential to virtually all biological processes. However, compared to nucleic acids, proteins and lipids, the knowledge regarding glycans has lagged behind mainly due to the structural diversity and rapid evolution of glycans, together with the limitations of analytical techniques. In this regard, discovery and development of new GBPs are of significance to basic, biotechnological and biomedical research.

### SUMMARY

Aspects of the disclosure relate to compositions and methods for expressing glycan binding proteins (GBPs) in a cell or cells. The disclosure is based, in part, on expression constructs engineered to express variants of Agaricus bisporus Ab-Y3 protein. As described further in the Examples section, Ab-Y3 has a unique binding specificity towards certain N-glycans with a Man₃GlcNAc₂ core structure and Gal-GlcNAc branches (e.g., bi-, tri- and tetra-antennary branches) that are commonly found in human cell line-expressed recombinant proteins. Recombinant Ab-Y3 variants described by the disclosure, in some embodiments, are useful for detection, isolation, purification, and screening of proteins having certain glycosylation patterns (e.g., certain immunoglobulin, IgG proteins).

Accordingly, in some aspects, the disclosure provides a recombinant Agaricus bisporus Y3 (Ab-Y3) protein variant comprising an amino acid sequence that is at least 70% identical to the amino acid sequence set forth in SEQ ID NO: 1, and comprises at least one amino acid substitution, insertion, or deletion relative to SEQ ID NO: 1.

In some embodiments, a recombinant Ab-Y3 protein variant lacks an N-terminal signal peptide domain (e.g., lacks one or more of amino acids 1-18 of SEQ ID NO: 1).

In some embodiments, a recombinant Ab-Y3 protein variant consists of the amino acid sequence set forth in SEQ ID NO: 3.

In some embodiments, a recombinant Ab-Y3 protein variant is glycosylated. In some embodiments, the glycosylation occurs at a position corresponding to N58 of SEQ ID NO: 1 (e.g., N40 of SEQ ID NO: 3) or at multiple sites (e.g., the protein is glycosylated at multiple amino acid residues). In some embodiments, a recombinant Ab-Y3 protein variant is unglycosylated (e.g., does not comprise any N-glycans).

In some embodiments, a recombinant Ab-Y3 protein variant binds to one or more types of complex N-glycans. In some embodiments, one or more complex N-glycans are one or more bi-antennary complex N-glycans. In some embodiments, one or more complex N-glycans are one or more tri-antennary complex N-glycans. In some embodiments, one or more complex N-glycans are one or more tetra-antennary complex N-glycans. In some embodiments, a recombinant Ab-Y3 protein variant binds to one or more N-glycans shown in FIG. 8.

In some aspects the disclosure provides a homo-oligomeric protein complex (e.g., a homo-oligomer) comprising between 2 and 10 protein subunits, wherein at least one of the subunits consists of the amino acid sequence set forth in SEQ ID NO: 3.

In some aspects, the disclosure provides an isolated nucleic acid comprising a nucleotide sequence encoding the recombinant Ab-Y3 protein variant as described herein. In some embodiments, the nucleotide sequence consists of nucleotides 55-408 of SEQ ID NO: 2.

In some aspects, the disclosure provides an isolated nucleic acid encoding a recombinant Ab-Y3 protein variant, wherein the isolated nucleic acid comprises a nucleotide sequence that is at least 80% identical to the nucleic acid sequence set forth in SEQ ID NO: 2, and comprises at least one nucleotide substitution, deletion, or insertion relative to SEQ ID NO: 2.

In some embodiments, a nucleotide sequence comprises nucleotides 55-408 of SEQ ID NO: 2. In some embodiments, a nucleotide sequence comprises a start codon (e.g., ATG) positioned 5′ relative to the nucleotide sequence encoding the recombinant Ab-Y3 protein variant.

In some embodiments, a nucleotide sequence is a codon-optimized nucleotide sequence. In some embodiments, a codon-optimized nucleotide sequence comprises the sequence set forth in SEQ ID NO: 4. In some embodiments, a codon-optimized nucleotide sequence comprises a 5′ start codon (e.g., ATG).

In some embodiments, an isolated nucleic acid further comprises a promoter operably linked to the nucleotide sequence encoding the recombinant Ab-Y3 protein variant. In some embodiments, a promoter is an inducible AOX1 promoter.

In some aspects, the disclosure provides a vector comprising an isolated nucleic acids described herein. In some embodiments, a vector is a plasmid, such as a bacterial plasmid vector.

In some aspects, the disclosure provides a host cell comprising an isolated nucleic acid vector as described herein. In some embodiments, a cell is a yeast cell, bacterial cell, or mammalian cell. In some embodiments, a yeast cell is a Pichia cell, such as a Pichia pastoris cell.

In some aspects, the disclosure provides a method for determining the presence of a target protein in a sample, the method comprising: contacting a sample with the recombinant Ab-Y3 protein variant as described herein under conditions sufficient for the recombinant Ab-Y3 protein variant to bind to one or more N-glycans in the sample; detecting binding of the recombinant Ab-Y3 protein variant to binding of one or more N-glycans in the sample; and determining that a target protein is present in the sample based upon detecting the binding of the recombinant Ab-Y3 protein variant to the one or more N-glycans.

In some embodiments, a target protein is a recombinantly expressed protein. In some embodiments, a target protein is recombinantly expressed in mammalian cells. In some embodiments, a target protein is expressed using a cell-free production method. In some embodiments, a target protein is an immunoglobulin (Ig) protein. In some embodiments, the target protein is IgG.

In some embodiments, detecting comprises performing a Western blot or a fluorescence-based N-glycan binding assay.

In some aspects, the disclosure provides a method for isolating an N-glycosylated target protein from a solution, the method comprising: contacting a solution comprising an N-glycosylated target protein with the Ab-Y3 protein variant as described herein under conditions sufficient for the recombinant Ab-Y3 protein variant to bind to one or more N-glycans of the target protein to form a complex; separating the complex from the solution; and dissociating the N-glycosylated target protein from the recombinant Ab-Y3 protein variant to form a pure target protein fraction.

In some embodiments, an N-glycosylated target protein is a recombinantly produced protein. In some embodiments, an N-glycosylated target protein is expressed in mammalian cells. In some embodiments, an N-glycosylated target protein is an immunoglobulin (Ig) protein. In some embodiments, the N-glycosylated target protein is IgG.

In some embodiments, a recombinant Ab-Y3 protein variant is bound to a solid substrate. In some embodiments, a solid substrate comprises one or more beads, a gel, or a resin.

In some embodiments, separating comprises physically separating the complex from the solution.

In some embodiments, dissociating comprises breaking one or more covalent bonds between a recombinant Ab-Y3 protein and a N-glycosylated target protein.

In some embodiments, a pure target protein fraction comprises at least 80%, 90%, 95%, 99%, 99.9% or 100% glycosylated target proteins (e.g., does not comprise un-glycosylated proteins).

### BRIEF DESCRIPTION OF THE DRAWINGS

FIG. 1 shows a protein sequence alignment of Ab-Y3 and Y3. Conserved cysteines are shaded. The amino acid sequence corresponding to the N-terminal signal peptide of each protein has been removed.

FIGS. 2A-2B show the codon-optimized nucleotide sequence of recombinant Ab-Y3 (FIG. 2A), and protein sequence of recombinant Ab-Y3 (FIG. 2B). A predicted N-glycosylation site is highlighted in yellow.

FIG. 3 shows SDS-PAGE analysis of wildtype Ab-Y3 with and without PNGase F treatment, and mutant protein Ab-Y3 N40Q.

FIG. 4 shows native gel analysis of wildtype Ab-Y3 protein.

FIG. 5 shows UV-vis spectral analysis of the Ellman's tests on Ab-Y3 protein (Agaricus; top curve), and cysteine (bottom).

FIG. 6 shows a standard curve of Glucose by phenol-sulfuric acid method.

FIG. 7 shows a hemagglutination assay of Ab-Y3 protein (Agaricus). Human O+ type (upper panel) and rabbit (lower panel) erythrocytes were used. Ab-Y3 solution (Agaricus) at 0.28 mM was serially diluted by two-fold (2 to 2048-times dilution).

FIG. 8 shows identification of top glycan ligands of Ab-Y3 by glycan array screening. Ab-Y3 was used at 50 m/mL concentration for the screening. Data are presented as mean±SD (n=6).

FIG. 9 shows a Western blot of Ab-Y3 with IgG.

FIGS. 10A-10C are schematics depicting structural features of Ab-Y3 protein. FIG. 10A shows the dimeric structure of Ab-Y3. Each monomer forms an αβα-sandwich. FIG. 10B shows Ab-Y3 and Y3 share a high degree structural similarity. FIG. 10C shows amino acid residues in the predicted glycan binding pocket of AbY3 share chemical similarity with Y3.

### DETAILED DESCRIPTION

Aspects of the disclosure relate to compositions and methods for expressing recombinant glycan binding proteins (GBPs) derived from Agaricus bisporus (e.g., Ab-Y3) in a cell. The disclosure is based, in part, on isolated nucleic acids engineered to express recombinant Ab-Y3 protein variants have unique binding specificity for complex N-glycans. Recombinant Ab-Y3 protein variants described herein are useful, in some embodiments, for isolating, purifying, detecting, or screening proteins comprising certain N-glycans (e.g., recombinant proteins glycosylated with bi-, tri-, and/or tetra-antennary N-glycans).

### Recombinant Ab-Y3 Protein Variants

In some aspects, the disclosure relates to recombinant proteins. As used herein, the term “recombinant” refers to a protein or peptide that has been expressed outside of its natural environment or artificially produced (e.g., by chemical synthesis, by recombinant DNA technology, etc.). For example, a recombinant protein may be heterologously expressed (e.g., expressed in a cell or organism that does not naturally express a version of that protein).

In some embodiments, a recombinant protein is a recombinant Ab-Y3 protein variant. Ab-Y3 is a glycan binding protein which is homologous to Coprinus comatus, Y3 protein. In some embodiments, a wild-type Ab-Y3 protein comprises the amino acid sequence set forth in NCBI Reference Sequence Number XP_007333380.1 (SEQ ID NO: 1). In some embodiments, a wild-type Ab-Y3 protein is encoded by a nucleic acid comprising the sequence set forth in NCBI Reference Sequence Number XM_007333318.1 (SEQ ID NO: 2).

As used herein, an “Ab-Y3 protein variant” refers to a protein derived from Agaricus bisporus Y3 glycan binding protein, which comprises and amino acid sequence having one or more genetic modifications relative to a wild-type Ab-Y3 protein and/or which is at least 85% identical to a wild-type Ab-Y3 protein. Thus, in some embodiments, an Ab-Y3 protein variant comprises an amino acid sequence that is at least 70%, at least 80%, at least 90%, at least 95%, or at least 99% identical to an amino acid sequence of a wild-type Ab-Y3 protein (SEQ ID NO: 1). In some embodiments, an Ab-Y3 protein variant is encoded by a nucleic acid sequence that is at least 70%, at least 80%, at least 90%, at least 95%, or at least 99% identical to a nucleic acid sequence encoding a wild-type Ab-Y3 protein (e.g., SEQ ID NO: 2). In some embodiments, a percentage identity is calculated by a global alignment-based algorithm. In some embodiments, a percentage identity is calculated by a local alignment-based algorithm.

An Ab-Y3 protein variant may comprise one or more genetic modifications relative to a wild-type Ab-Y3 protein. As used herein, the term “genetic modification” refers to amino acid substitution (conservative, missense and/or non-sense), deletion and/or insertion. Thus in some embodiments, a recombinant Ab-Y3 protein variant comprises an amino acid sequence having at least 1, at least 2, at least 3, at least 4, at least 5, at least 10, at least 20, at least 30, at least 40, at least 50, at least 75, or at least 100 genetic modifications relative to wild-type Ab-Y3 protein (e.g., SEQ ID NO: 1). In some embodiments a recombinant Ab-Y3 protein variant is truncated relative to wild-type Ab-Y3 (e.g., SEQ ID NO: 1). Truncations may occur at the N-terminus or C-terminus of the portion of Ab-Y3. For example, a recombinant Ab-Y3 protein variant may be truncated by 1, 2, 3, 4, 5, 10, 20, 30, 40, 50, 75, 100 or 200 amino acids at it N-terminus or C-terminus relative to wild-type Ab-Y3 (e.g., SEQ ID NO: 1). In some embodiments, a recombinant Ab-Y3 protein variant comprises a truncation that results in the recombinant Ab-Y3 protein lacking one or more amino acids of an Ab-Y3 signal peptide (e.g., as set forth in amino acid residues 1-18 of SEQ ID NO: 1). In some embodiments, a recombinant Ab-Y3 protein variant lacks amino acids corresponding to amino acid residues 1-18 of SEQ ID NO: 1.

A recombinant Ab-Y3 protein variant that is truncated relative to a wild-type Ab-Y3 protein may have an amino acid sequence that comprises or lacks a start codon (ATG). In some embodiments, an amino acid sequence encoding a recombinant Ab-Y3 protein variant lacks a start codon (e.g., ATG, AUG), and thus protein expression is initiated from a vector sequence (e.g., a pUC57 vector sequence). In some embodiments, an amino acid sequence encoding a recombinant Ab-Y3 protein variant is modified to include a methionine (M) at the N-terminus of the coding sequence (e.g., the truncated Ab-Y3 protein coding sequence). In some embodiments, the methionine is encoded by a start codon. In some embodiments, the start codon is ATG. In some embodiments, the start codon is an alternative start codon, for example CUG, GUG, UUG, etc.).

Recombinant Ab-Y3 variants as described herein may form oligomeric complexes (oligomers). In some embodiments, an oligomeric complex (e.g., oligomer) comprises at least 2 (e.g., 2 or more) protein subunits, for example 2, 3, 4, 5, 6, 7, 8, 9, 10, 20, 30, 50, or 100 protein subunits. In some embodiments, an oligomeric complex (e.g., oligomer) comprises between 2 and 20 (e.g., 2, 3, 4, 5, 6, 7, 8, 9, 10, 11, 12, 13, 14, 15, 16, 17, 18, 19, or 20) protein subunits, wherein at least one of the subunits is a recombinant Ab-Y3 protein variant. In some embodiments, an oligomeric complex (e.g., oligomer) comprises between 2 and 10 protein subunits, wherein at least one of the subunits is a recombinant Ab-Y3 protein variant. In some embodiments, the complex is a homo-oligomeric complex (e.g., all subunits of the complex have identical amino acid sequences). In some embodiments, the complex is a hetero-oligomeric complex (e.g., at least two subunits of the complex do not have identical amino acid sequences).

Aspects of the disclosure relate to recombinant Ab-Y3 protein variants (or a complex comprising one or more Ab-Y3 protein variants) that retain the capability to bind to certain complex N-glycans. “Complex N-glycans” refers to oligosaccharide moieties comprising a Man₃GlcNAc₂ core structure and two or more (e.g., 2, 3, or 4) antennary glycan structures, for example as described by Stanley et al. “N-Glycans”. In: Varki et al., editors. Essentials of Glycobiology. 2nd edition. Cold Spring Harbor (NY): Cold Spring Harbor Laboratory Press; 2009. Chapter 8. Examples of complex glycans include but are not limited to Galb1-4GlcNAcb1-6(Galb1-4GlcNAcb1-2)Mana1-6(Gabb1-4GlcNAcb1-2Mana1-3)Manb1-4GlcNAcb1-4(Fuca1-6)GlcNAcb; Galb1-4GlcNAcb1-2Mana1-6(Galb1-4GlcNAcb1-2Mana1-3)Mana1-4GlcNAcb1-4GlcNAcb; and Galb1-4GlcNAcb1-6(Galb1-4GlcNAcb1-2)Mana1-6(Galb1-4GlcNAcb1-2)Mana1-3-Manb1-4GlcNAcb1-4GlcNAcb, etc. In some embodiments, a recombinant Ab-Y3 protein variant binds to a complex N-glycan that results from recombinant expression of a protein in a mammalian cell, for example a human cell or a Chinese hamster ovary (CHO) cell. Examples of complex N-glycans produced during recombinant protein expression in mammalian cells are described, for example by Hossler et al. (2009) Glycobiology 19(9):936-949. In some embodiments, recombinant Ab-Y3 protein variants described herein bind to N-glycans comprising a terminal sialic acid (e.g., Neu5Ac) with a lower binding affinity than binding to N-glycans that lack a terminal sialic acid (Neu5Ac). In some embodiments, the lower binding affinity for N-glycans comprising a terminal Neu5Ac is 2-fold, 3-fold, 5-fold, 10-fold, 50-fold, 100-fold, or 1000-fold less than the binding affinity for N-glycans lacking a terminal Neu5Ac. In some embodiments, the lower binding affinity for N-glycans comprising a terminal Neu5Ac is more than 1000-fold lower than the binding affinity for N-glycans lacking a terminal Neu5Ac.

In some embodiments, recombinant Ab-Y3 protein variants (or a complex comprising one or more Ab-Y3 protein variants) bind to one or more complex N-glycans of a glycosylated protein, for example an immunoglobulin (Ig) protein. Examples of Ig proteins include but are not limited to IgA, IgD, IgE, IgG, and IgM. In some embodiments a recombinant Ab-Y3 protein variant binds to an IgG protein (e.g., a glycosylated IgG protein).

Methods of genetically modifying recombinant Ab-Y3 protein variants and screening for retention of functional activity are known in the art and available to the skilled artisan. For example, a recombinant Ab-Y3 protein variant may be modified by directed evolution or random mutagenesis and biochemically assayed for the capability to bind certain glycans (e.g., complex N-glycans, such as bi-, tri-, and tetra-antennary N-glycans.

In some embodiments the disclosure relates to isolated nucleic acids and expression constructs encoding a recombinant Ab-Y3 protein variant. As used herein “nucleic acid” refers to a DNA or RNA molecule. Nucleic acids are polymeric macromolecules comprising a plurality of nucleotides. In some embodiments, the nucleotides are deoxyribonucleotides or ribonucleotides. In some embodiments, the nucleotides comprising the nucleic acid are selected from the group consisting of adenine, guanine, cytosine, thymine, uracil and inosine. In some embodiments, the nucleotides comprising the nucleic acid are modified nucleotides. Non-limiting examples of natural nucleic acids include genomic DNA and plasmid DNA. In some embodiments, the nucleic acids of the instant disclosure are synthetic. As used herein, the term “synthetic nucleic acid” refers to a nucleic acid molecule that is constructed via the joining nucleotides by a synthetic or non-natural method. One non-limiting example of a synthetic method is solid-phase oligonucleotide synthesis. In some embodiments, the nucleic acids of the instant disclosure are isolated. In some embodiments, a nucleic acid sequence encoding an Ab-Y3 protein variant is a codon-optimized nucleic acid sequence.

In some aspects, the disclosure relates to an expression construct comprising a recombinant Ab-Y3 protein variant as described by the disclosure. As used herein, the term “expression construct” refers to an artificially constructed molecule comprising a nucleic acid (e.g., DNA) capable of artificially carrying foreign genetic material into another cell (for example, a bacterial cell, yeast cell, etc.). In some embodiments, an expression construct comprises one or more regulatory sequences that are operably linked to a nucleic acid sequence encoding a recombinant Ab-Y3 protein variant. As used herein, “operably linked” means that the control sequence(s) is in the correct location and orientation in relation to the nucleic acid to control RNA polymerase initiation and expression of the gene. Examples of regulatory sequences include but are not limited to promoters, enhancer sequences, internal ribosomal entry sites, and polyA regions. In some embodiments, an expression construct comprises a promoter that is operably linked to a nucleic acid sequence encoding a recombinant Ab-Y3 protein variant. A promoter may be a constitutive promoter, inducible promoter, or a tissue-specific promoter.

An isolated nucleic acid or expression construct may be contained on a vector. In some embodiments, vectors carry common functional elements including an origin of replication, a multicloning site, a selectable marker and optionally a promoter sequence. In some embodiments, the selectable marker is a bacterial resistance gene, for example kanamycin, chloramphenicol or 62 -lactamase. Non-limiting examples of vectors include plasmids, viral vectors, cosmids, and artificial chromosomes. In some embodiments, the vector is a high-copy plasmid. In some embodiments, the vector is a low-copy plasmid. In some embodiments, the vectors of the disclosure are maintained inside cells. In some embodiments, the vectors of the disclosure are maintained in a non-cellular environment, for example as part of a kit. Methods of introducing vectors into bacteria are well known in the art and described, for example, in Current Protocols in Molecular Biology, Ausubel et al. (Eds), John Wiley and Sons, New York, 2007.

The disclosure relates, in part, to host cells comprising isolated nucleic acids, expression constructs, and vectors as described herein. A host cell may be any cell that contains the components required for expressing a recombinant Ab-Y3 protein variant, for example a mammalian cell, bacterial cell, yeast cell, insect cell, etc. In some embodiments, a host cell is a yeast cell. Examples of yeast cells include but are not limited to Saccharomyces cerevisiae cells, Pichia pastoris cells, Kluyveromyces cells, Hansenula cells, and Yarrowia cells. Examples of bacterial cells include but are not limited to E. coli cells and Bacillus cells. Examples of mammalian cells include but are not limited to human embryonic kidney cells (HEK cells, such as HEK293 cells), HeLa cells, Chinese hamster ovary (CHO) cells, and COS-7 cells.

### Methods

Without wishing to be bound by any particular theory, the unique glycan binding specificity of recombinant Ab-Y3 protein variants described by the disclosure is useful for binding certain glycoproteins (e.g., complex N-glycans). Accordingly, aspects of the disclosure relate to methods for detecting, isolating, purifying, and/or screening for certain proteins (e.g., glycosylated proteins having certain complex N-glycans). In some embodiments, methods described by the disclosure comprise contacting an N-glycan (e.g., protein containing one or more N-glycans) or a solution comprising one or more glycosylated proteins, with a recombinant Ab-Y3 protein variant as described herein.

In some aspects, the disclosure provides a method for determining the presence of a target protein in a sample, the method comprising: contacting a sample with the recombinant Ab-Y3 protein variant as described herein under conditions sufficient for the recombinant Ab-Y3 protein variant to bind to one or more N-glycans in the sample; detecting binding of the recombinant Ab-Y3 protein variant to binding of one or more N-glycans in the sample; and determining that a target protein is present in the sample based upon detecting the binding of the recombinant Ab-Y3 protein variant to the one or more N-glycans.

In some aspects, the disclosure provides a method for isolating an N-glycosylated target protein from a solution, the method comprising: contacting a solution comprising an N-glycosylated target protein with the Ab-Y3 protein variant as described herein under conditions sufficient for the recombinant Ab-Y3 protein variant to bind to one or more N-glycans of the target protein to form a complex; separating the complex from the solution; and dissociating the N-glycosylated target protein from the recombinant Ab-Y3 protein variant to form a pure target protein fraction.

As used herein, a “target protein” refers to a protein comprising one or more complex N-glycans. In some embodiments, a target protein is a recombinantly expressed protein (e.g., a protein that is heterologously expressed in a mammalian cell, such as a human cell). In some embodiments, a target protein is expressed by a cell-free protein synthesis method, for example as described by Lee et al. (2018) FEMS Microbiology Letters 365(17):fny174. In some embodiments, a target protein is a therapeutic protein. A “therapeutic protein” refers to a protein characterized by a biological function or activity that renders it useful for treating a particular disease, condition, or disorder. Examples of therapeutic proteins include but are not limited to antibodies (e.g., anti-cancer antigen-specific antibodies, etc.), peptides (e.g., cytotoxic peptides, etc.), kinases (e.g., MAPK, etc.), kinase inhibitors (e.g., mTOR inhibitors, etc.), etc. In some embodiments, a target protein is an immunoglobulin (Ig) protein, or a protein comprising an Ig protein, such as an antibody. In some embodiments, the Ig protein is IgG.

One or more target proteins may be present in a sample or a solution. Examples of samples include biological samples (e.g., blood samples, tissue samples, cell samples, CNS fluid samples, saliva samples, urine samples, etc.) and laboratory-prepared samples (e.g., cell lysates, purified cellular material, eluates, etc.). In some embodiments, a sample is obtained from a subject, for example a mammalian subject such as a human. “Solutions” generally refers to aqueous solutions comprising a target protein and one or more additional components, for example water, buffers, other proteins (e.g., other proteins that are un-glycosylated or have a hybrid glycosylation pattern), nucleic acids, small molecules, etc.

Detection of recombinant Ab-Y3 protein variant binding to N-glycans can be carried out by any suitable method. In some embodiments, the binding is detected by a Western blot (e.g., a Western blot using an anti-Ab-Y3 antibody, or an antibody specific for the target protein). In some embodiments, the binding is detected by a fluorescence-based N-glycan binding assay. For example, in some embodiments, a biotin-labeled recombinant Ab-Y3 protein variant is tagged with biotin and incubated in a solution containing a target protein; subsequently, a fluorescently labeled streptavidin (e.g., Streptavidin-488) is added to the solution and relative fluorescence is quantified (e.g. relative to a control sample that does not contain target N-glycan or target protein or relative to a standard curve). In some embodiments, detection is performed using a chromatographic method (e.g., HPLC) or a spectrometry-based method (e.g., LC/MS, MS/MS, etc.).

In some aspects, binding of a recombinant Ab-Y3 protein variant to one or more complex N-glycans in a solution allows for isolation (e.g., separation) of complexes comprising the Ab-Y3 protein variant bound to the protein(s) comprising the one or more complex N-glycans from other components of a solution. In some embodiments, the separation is performed by chromatographic methods. Examples of chromatographic methods include but are not limited to affinity chromatography (e.g., column chromatography, planar chromatography), gas chromatography, liquid chromatography (e.g., HPLC, etc.), ion exchange chromatography, size-exclusion chromatography, etc. In some embodiments, the separation is performed by affinity chromatography. In some embodiments, the separation is performed by spectrometry-based methods (e.g., LC/MS, MS/MS, MS-TOF, etc.).

Affinity chromatography refers to biochemical methods of separating a mixture based on specific binding interactions between cognate binding partners (e.g., an antibody and an antigen, enzyme and substrate, protein and ligand, etc.). Generally, one binding partner is attached to a solid substrate, for example beads, resin, gel, or other types of solid matrix. Thus, in some embodiments, a recombinant Ab-Y3 protein described herein is bound (e.g., adsorbed, conjugated, covalently or non-covalently connected, etc.) to a solid substrate. In some embodiments, a recombinant Ab-Y3 protein (e.g., a recombinant Ab-Y3 protein on a solid support) and one or more complex N-glycans (e.g., one or more N-glycans of a target protein) interact or bind to form a complex (e.g., a protein complex).

After separating a complex from the remaining components of a solution, the recombinant Ab-Y3 protein variant is dissociated from the N-glycan containing protein(s). “Dissociation” or “dissociating” generally refers to physically separating the components of a complex (e.g. a complex comprising a recombinant Ab-Y3 protein bound to a complex N-glycan, such as one or more complex N-glycans of a target protein) from one another. In some embodiments, dissociation comprises breaking one or more covalent bonds between a recombinant Ab-Y3 protein and an N-glycosylated target protein. For example, in some embodiments dissociation comprises changing one or more physiochemical characteristics of the environment surrounding the complex (e.g., an affinity column comprising the complex) such that the bonds between the recombinant Ab-Y3 protein variant and the target protein (e.g., one or more complex N-glycans of the target protein) are broken. Examples of physiochemical characteristics include but are not limited to salt concentration, pH, temperature, etc. In some embodiments, dissociating a recombinant Ab-Y3 protein variant from the one or more N-glycans of a target protein comprises eluting the target protein from an affinity column (e.g., a solid substrate comprising recombinant Ab-Y3 protein variants).

In some embodiments, dissociating the target protein (e.g., complex N-glycan containing protein) from the complex (e.g., from the recombinant Ab-Y3 protein variant) produces a pure target protein fraction. A pure target protein fraction refers to a composition (e.g., a solution, such as an aqueous solution) comprising at least 70%, at least 80%, at least 90%, at least 95%, at least 99%, at least 99.9%, or 100% (no or substantially no proteins other than the target protein) target proteins.

### EXAMPLES

### Example 1

This example describes biochemical and structural characterization of recombinant Ab-Y3, which is derived from an Agaricus bisporus fungal glycan binding protein (GBP). Recombinant Ab-Y3 was expressed and characterized as a glycoprotein which comprises multiple disulfide bridges. Screening of Ab-Y3 in a mammalian glycan microarray indicated a unique glycan binding specificity for Ab-Y3 towards glycans with Man₃GlcNAc₂ core structure and Gal-GlcNAc branches that are commonly found in human cell line-expressed recombinant proteins. Analysis of Ab-Y3 crystal structure revealed a single-domain αβα-sandwich motif, and two monomers that form a large dimer with 10-stranded, antiparallel β-sheet flanked by α-helices on each side. The structure of Ab-Y3 and its homolog in Coprinus comatus, Y3 protein, vary in their β1α1 loop regions nearby the binding pockets, which may account for differences in glycan specificity of the proteins.

### Expression and Characterization of Recombinant Ab-Y3 Protein

A codon-optimized gene encoding full-length Ab-Y3 was synthesized (GenScript, USA), and cloned into XhoI/NotI sites of pPICZaA vector (ThermoFisher Scientific). Pichia pastoris X-33 was transformed with a Sad linearized pPICZαA-AbY3 plasmid. After Zeocin selection, multiple single colonies were randomly picked to access the Ab-Y3 production in small scale culture (10 mL). The strain with the highest yield was selected for scaled expression of Ab-Y3. One liter of YPD (1% yeast extract, 2% peptone, and 2% glucose) was inoculated with 20 mL of saturated Ab-Y3 culture in YPD and grown (˜24 h, 30° C., 250 rpm) to OD600≈12-18. The culture was centrifuged at 2000×g (10 min) and resuspended in 200 mL of BMMY (100 mM potassium phosphate, pH 6.0, 1.34% YNB, 4×10-5 biotin, and 0.5% methanol). The cells were grown in BMMY media at 30° C. with shaking. Methanol was added to a final concentration of 0.5% to maintain induction every day. After 3 days, the culture supernatant was harvested by centrifugation (3000×g, 10 min), filtered and dialyzed against buffer (150 mM NaCl, and 20 mM Tris-HCl, pH 7.5). The supernatant was further concentrated, and purified by gel filtration chromatography (HiLoad 16/60 SuperDex-75 column, AKTA FPLC System, GE Healthcare) with 150 mM NaCl, and 20 mM Tris-HCl, pH 7.5. Purified Ab-Y3 was concentrated to 10 mg·mL⁻¹. The protein concentration was determined by Bradford assay (BSA as control) and the purity was evaluated by SDS-PAGE.

There is one predicted N-glycosylation site in recombinant Ab-Y3 protein (Asn40, N40; corresponding to N58 in SEQ ID NO: 1). To confirm the glycosylation, a mutant Ab-Y3-N40Q clone was constructed by using site-directed mutagenesis. The pPICZaA-Ab-Y3 plasmid was used as a template for site-directed mutagenesis PCR with the point-mutation primers (Ab-Y3_N40Q Fw: 5′-GTATTCAGGATAGCGTTA-3′ (SEQ ID NO: 5), Ab-Y3_N40Q Rv: 5′-CTATCCTGAATACGCACT-3′ (SEQ ID NO: 6)). After DpnI digestion, the PCR fragments were transformed into E. coli DH5a. The colonies were selected on low salt LB agar medium containing 25 μg/ml of Zeocin, and the plasmids were purified with Zyppy™ Plasmid Miniprep Kit. Sequence-confirmed pPICZaA-Ab-Y3-N40Q construct was then transformed into Pichia pastoris X-33 and expressed to mutant Ab-Y3-N40Q protein using above described methods.

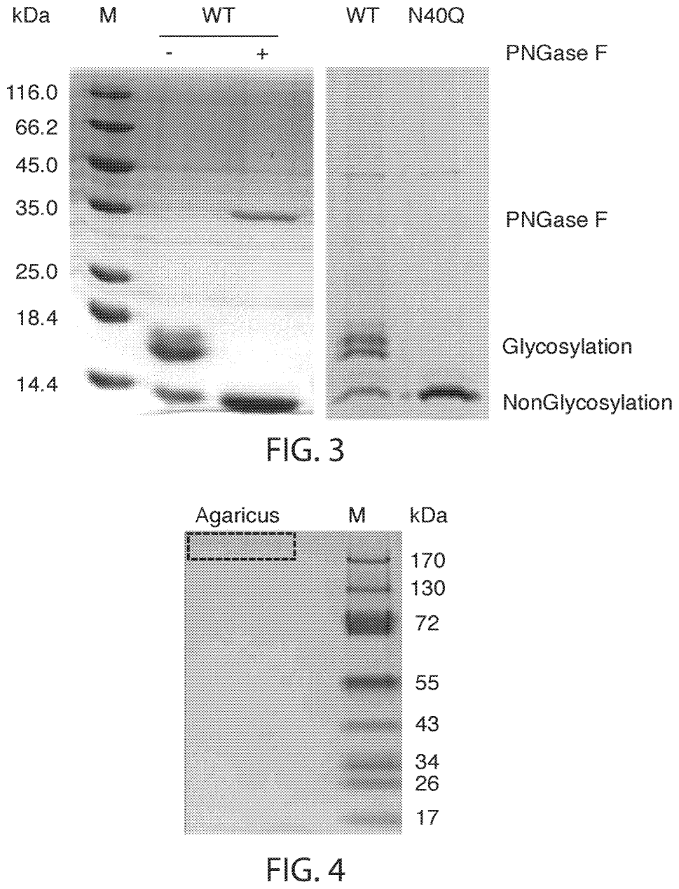

Given the putative post-translational modification of N-glycosylation site at Asn40, a yeast expression system was chosen to express Ab-Y3 protein. A codon-optimized Ab-Y3 coding sequence for yeast codon usage was generated in order to achieve high-yield expression of Ab-Y3. The gene and protein sequence of recombinant Ab-Y3 are shown in FIGS. 2A-2B. The yield of recombinant Ab-Y3 was about 20 mg/L. The purified Ab-Y3 protein was separated by SDS-PAGE and showed three bands of approximate 12, 15 and 16 kDa (FIG. 3). The two upper bands were glycosylated. Treatment with PNGase F, which removes N-linked glycans, resulted in a single band at ˜12 kDa (FIG. 3) that corresponds to the calculated molecular weight of 12293.8. Therefore, recombinant Ab-Y3 expressed in P. pastoris was partially glycosylated with two different patterns. To demonstrate the putative glycosylation site at Asn40, the asparagine residue at position 40 (N40) was mutated to glutamine (N40Q). The mutant protein was then expressed.

Since the homolog of Ab-Y3 protein, Y3 protein, was determined to be a dimer in native state, the oligomeric state of Ab-Y3 protein was evaluated by native gel analysis. Ab-Y3 protein was observed to be larger than 170 kDa (FIG. 4), indicating Ab-Y3 protein to be a homo-oligomer in native state.

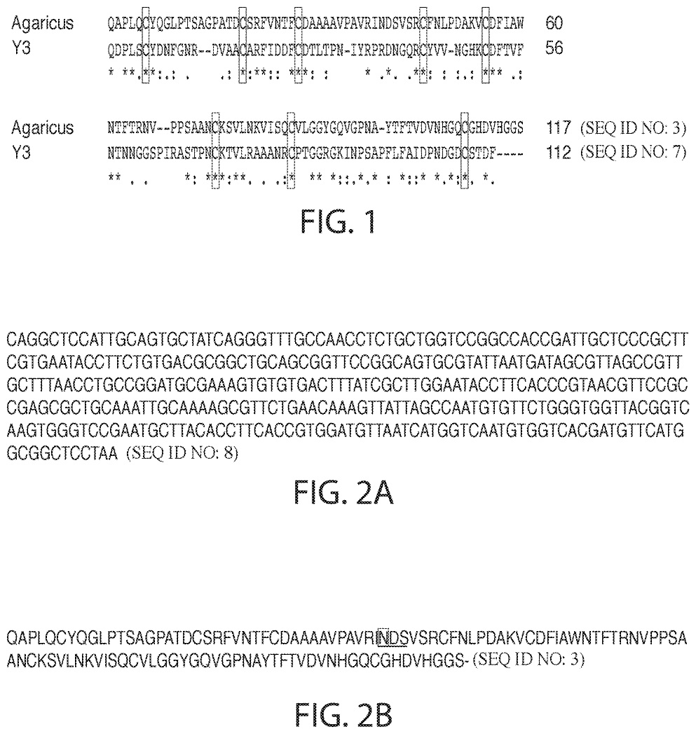

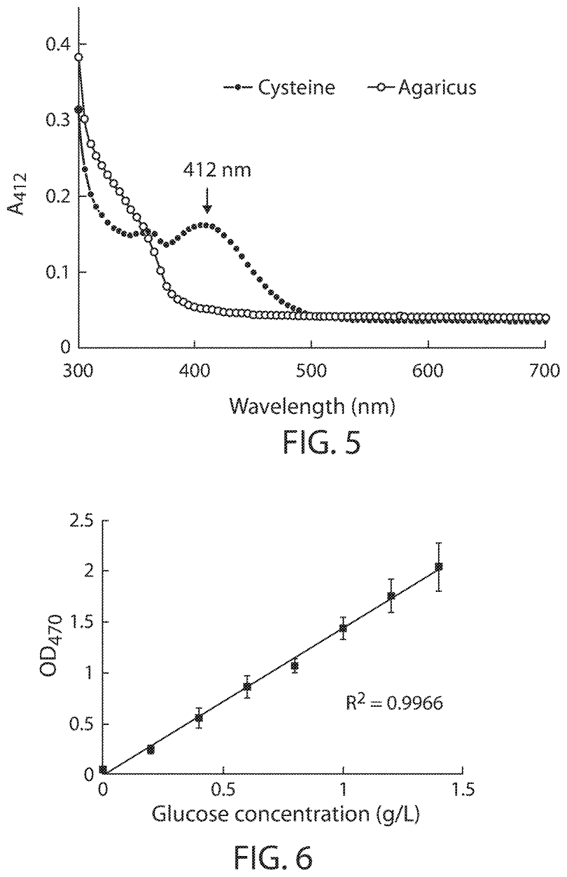

Protein sequence alignment of Ab-Y3 and Y3 indicated that all cysteine residues are highly conserved in both proteins (FIG. 1). In previous study of Y3, it was observed that these eight cysteine residues form four intramolecular disulfide bridges, which contribute to the stability of Y3. To investigate if cysteines in Ab-Y3 form disulfide bonds, Ellman's test for measuring free thiol groups was performed. Briefly, ten microliters of Ab-Y3 (100 μM) or L-cysteine (100 μM, positive control) was mixed with 990 μL of DTNB reagent (100 μM, 0.1M Tris, pH 8.0, ThermoFisher Scientific), and incubated at room temperature for 10 min. The optical absorbance was measured and recorded at range of 300-700 nm. By recording the absorbance varying from 300 to 700 nm, no typical absorbance peak of -SH was observed at wavelength 412 nm (FIG. 5), excluding the presence of any free thiol in Ab-Y3 protein.

### Glycan Modification and Content of Ab-Y3

Recombinant Ab-Y3 protein was determined to be a glycoprotein. The carbohydrate content in Ab-Y3 protein was then assessed by phenol sulfuric acid analysis. Briefly, a standard curve was established with D-Glucose (0 to 1.4 g/L) and protein solutions (50 μL) were added to flat-bottom microplate followed by the rapid mixing with 150 μL of concentrated sulfuric acid. After gentle shaking to achieve homogenized solution, 30 μL of 5% phenol was added and the plate was kept at room temperature for 10 min. Protein free buffer and D-Glucose were used as controls. Absorbance was recorded at 490 nm by microplate reader (BioTek). Carbohydrate content (%) was then calculated using the equation of raw carbohydrate content (mg/mL)/protein concentration (mg/mL) determined by Bradford assay×100%. All experiments were independently repeated at least three times.

A linear, standard curve was generated by gradient concentrations of D-Glucose (0 to 1.4 g/L) (FIG. 6). The total of carbohydrates in the Ab-Y3 sample was calculated to be 123.141±5.1796% (wt/wt). The large amount of carbohydrate contents indicates that, aside from post-translational glycosylation, Ab-Y3 protein may interact and be co-purified with carbohydrates either from induction medium or yeast endogenous sugars. A mutation at predicted N-glycosylation site (N40Q) was then created to examine the ratio of glycan modification and binding.

### Hemagglutination Activity of Ab-Y3

Agglutinating activity of Ab-Y3 protein was examined by using both human type O+ and rabbit red blood cells (Innovative research, USA). A serial twofold dilution of the Ab-Y3 solution (starting concentration 0.28 mM) in microtiter U-plates (50 μL) was mixed with 50 μL of 2% suspension of red blood cells in 0.01 M PBS (pH 7.4) at room temperature. Protein free buffer was used as controls. The results were recorded when the blank was fully sedimented.

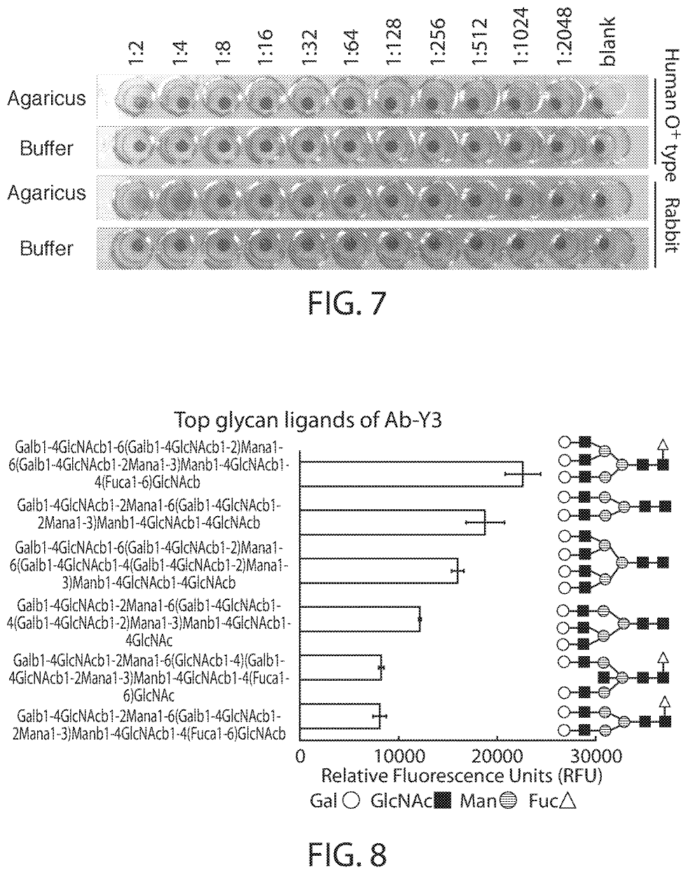

Lectins have been characterized to agglutinate erythrocytes by recognizing a carbohydrate on the cell surface and forming a cross-linked network in suspension. Ab-Y3 protein was observed to agglutinate rabbit red blood cell at concentration of 0.07 mM but did not agglutinate human erythrocytes at concentration up to 0.14 mM (FIG. 7), which is strikingly different from the typical function of other lectins, but similar to its homolog Y3.

### Glycan Binding Profile of Ab-Y3

It was previously observed that Y3 is a glycan binding protein with a unique specificity toward a trisaccharide, GalNAcβ1-4(Fucα1-3)GlcNAc (LDNF). Here, the glycan binding specificity of its homolog, Ab-Y3, is described.

A glycan microarray analysis was performed using a mammalian printed array (version 5.3, 600 glycans in six replications). Ab-Y3 was biotinylated using EZ-Link NHS-PEG4-Biotinylation Kit (ThermoFisher Scientific). Biotin-labeled Ab-Y3 at 5 and 50 μg/mL was analyzed. Streptavidin-488 was used to detect biotinylated Ab-Y3 that bound to the glycans on the array. The average binding for each glycan target as well as SD was calculated after the highest and lowest point from each set of six replicates were removed, and glycans were then ranked and sorted. The scanner response was linear to a maximal relative fluorescence unit value of about 50,000.

IgG binding to Ab-Y3 was also investigated. Increasing amounts of Ab-Y3 (2, 4, 6, 8 μg) and control protein bovine serum albumin (BSA, 2, 4, 6 μg) were loaded to 12% SDS-PAGE. After electrophoresis, proteins were transferred to PVDF membranes, blocked for 1 h with 5% fat free milk at room temperature, and incubated with HRP-conjugated anti-mouse IgG antibody (1:1000 dilution, Cell Signaling Technology). Following a wash step with Tris buffered saline/Tween-20, the binding complexes were detected using the standard electrochemiluminescence method.

After conducting glycan microarray screening of Ab-Y3 against 600 mammalian glycans, it was observed that Ab-Y3 exclusively recognized a panel of complex bi-, tri- and tetra-antennary glycans with Man₃GlcNAc₂ core structure and Gal-GlcNAc branches (FIG. 8). Such glycan structures with Neu5Ac attached to Gal via α-2,3 linkages are typical to recombinant glycoproteins expressed by human cell lines, for example recombinant immunoglobulin G (IgG), and human erythropoietin (rhEPO) expressed by Chinese hamster ovary (CHO) cells. However, the terminal N-acetylneuraminic acid (Neu5Ac) significantly lowers the binding affinities of glycans to Ab-Y3. Neu5Ac is the primary sialic acid found in human glycoproteins.

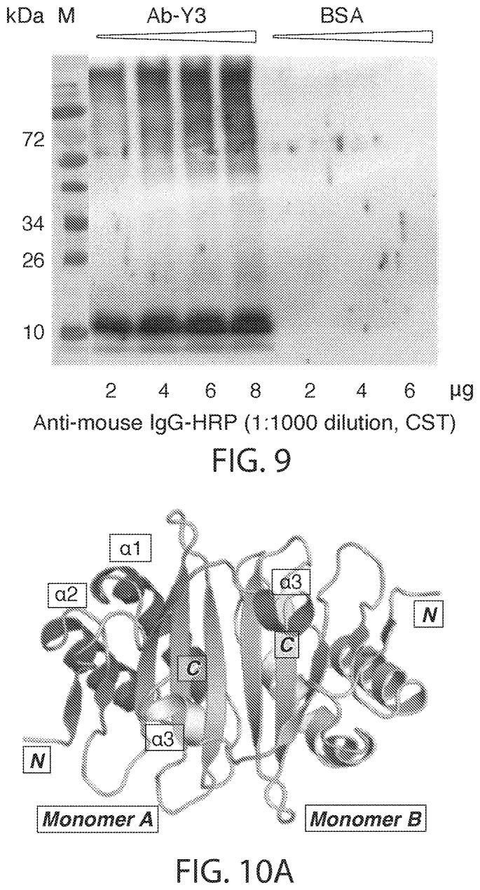

Since IgG consists of a biantennary core of N-acetylglucosamine and mannose with added terminal and branching carbohydrate residues such as N-acetylglucosamine, fucose, sialic acid, and galactose, it was investigated whether Ab-Y3 is able to bind IgG. Ab-Y3 and control protein BSA were firstly immobilized on PVDF membrane and then incubated with recombinant horseradish peroxidase (HRP)-linked IgG overnight. In the presence of a substrate, HRP produced the detectable signal that indicates the location of Ab-Y3 (FIG. 9), therefore demonstrates the interaction of Ab-Y3 to IgG. In contrast, signal was missing in the lanes of control protein BSA (FIG. 9), which further demonstrated the binding specificity of Ab-Y3 to IgG.

### AbY3 is Structurally Similar to Y3.

Structural studies of Ab-Y3 were performed. Ab-Y3 was crystallized utilizing vapor diffusion sitting drop technique screening against a variety of commercial screens. At the 1:1 ratio, protein (5 mg/mL) and precipitant solutions were mixed (1 μL) and equilibrated against 60 μL of reservoir solution. Rod shaped crystals were obtained with dimensions 270×30×30 μm using 0.1M Tris pH 8.5, 25% w/v Polyethylene glycol 3,350 after incubation at 25° C. for 5 months. Crystals were transferred to cryoprotectant (0.1M Tris pH 8.5, 25% w/v Polyethylene glycol 3,350, 30% glycerol) and allowed to equilibrate for 5 minutes, then flash frozen in liquid nitrogen.

X-ray diffraction data sets of Ab-Y3 crystals were collected at a wavelength of 1.033 Å at 100K. The XDS software package was utilized to merge datasets, and AIMLESS, from CCP4 program suite, was utilized to scale to space group P 21212 at 1.02 Å resolution.

The structure was solved using molecular replacement with Y3 from Coprinus comatus (PDB: 5V6J) as the search model, using Phaser from the CCP4 program suite. Model building was initiated using BUCCANEER and completed with iterative rounds of manual rebuilding and refinement using COOT and refmac/PHENIX.REFINE, respectively. CHES buffer molecules were manually added using COOT after careful inspection of the density maps. All programs used were from the CCP4 programs suite or Phenix software suite.

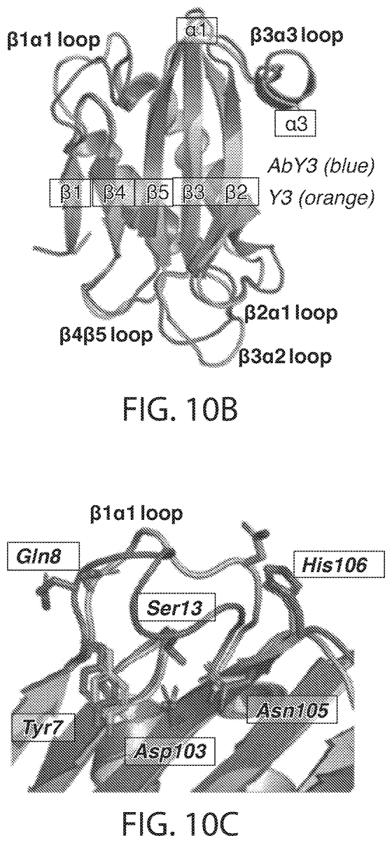

The crystal structure of Ab-Y3 was solved to a resolution of 1.02 Å. It was observed that each AbY3 monomer forms an αβα-sandwich, containing a five-stranded antiparallel β-sheet with two α helices on one face and a short α helix on the opposite face, also known as a Rossmann-like fold. Two monomers dimerize to form a large, ten-stranded antiparallel β-sheet with equivalent helices on the same face (FIGS. 10A-10C). All eight cysteine residues form intramonomer disulfide bonds, owing to its stability; Cys6-Cys85, Cys20-Cys55, Cys28-Cys74, and Cys46-Cys109 all form disulfide bonds that have clearly observed electron density in the x-ray structure. A large glycan binding pocket was identified near the β1α1 loop, sitting above beta strands 1, 4, and 5. Key amino acids in this region include Tyr7, Gln8, Ser13, Asp103, Asn105, and His106. Y3 has the same αβα-sandwich scaffold and its predicted binding pocket contains chemically similar amino acids at equivalent positions: Tyr25, Asp26, Asn30, Asp120, Asn122, and Asp123, respectively (FIG. 10B). These amino acids directly interact with their glycan targets, and the variation at these important positions reveals the evolutionary means by which the two members of the same GBP family bind their unique substrates.

SEQUENCES

>NCBI Reference Sequence Number XP_007333380.1;
Wild-type Agaricus bisporus amino acid sequence
(SEQ ID NO: 1); signal peptide underlined
MFSKVYLVASTLIAVAVAQAPLQCYQGLPTSAGPATDCSRFVNTFCDAA
AAVPAVRINDSVSRCFNLPDAKVCDFIAWNTFTRNVPPSAANCKSVLNK
VISQCVLGGYGQVGPNAYTFTVDVNHGQCGHDVHGGS
>NCBI Reference Sequence Number XM_007333318.1;
Wild-type Agaricus bisporus nucleic acid sequence
(SEQ ID NO: 2); signal peptide underlined
ATGTTTTCCAAAGTCTATCTTGTTGCATCTACTCTCATCGCTGTTGCGG
TGGCTCAAGCACCTCTCCAGTGCTACCAAGGCTTACCCACTTCTGCGGG
TCCCGCAACTGACTGTTCCCGATTTGTGAACACCTTTTGCGACGCGGCT
GCCGCTGTCCCTGCGGTACGCATTAACGATTCAGTCAGTCGTTGCTTCA
ATTTACCAGATGCCAAAGTTTGCGATTTCATTGCGTGGAACACATTCAC
GCGCAACGTTCCGCCTTCAGCGGCTAATTGCAAATCCGTGCTGAACAAA
GTTATTTCTCAATGCGTATTGGGAGGTTATGGACAGGTTGGGCCCAATG
CGTATACTTTCACGGTTGACGTCAACCATGGACAGTGTGGCCATGATGT
CCATGGTGGATCCTAG
>Recombinant Ab-Y3 amino acid sequence (SEQ ID
NO: 3)
QAPLQCYQGLPTSAGPATDCSRFVNTFCDAAAAVPAVRINDSVSRCFNL
PDAKVCDFIAWNTFTRNVPPSAANCKSVLNKVISQCVLGGYGQVGPNAY
TFTVDVNHGQCGHDVHGGS
>Codon-optimized recombinant Ab-Y3 nucleic acid
sequence (SEQ ID NO: 4)
CAGGCTCCATTGCAGTGCTATCAGGGTTTGCCAACCTCTGCTGGTCCGG
CCACCGATTGCTCCCGCTTCGTGAATACCTTCTGTGACGCGGCTGCAGC
GGTTCCGGCAGTGCGTATTAATGATAGCGTTAGCCGTTGCTTTAACCTG
CCGGATGCGAAAGTGTGTGACTTTATCGCTTGGAATACCTTCACCCGTA
ACGTTCCGCCGAGCGCTGCAAATTGCAAAAGCGTTCTGAACAAAGTTAT
TAGCCAATGTGTTCTGGGTGGTTACGGTCAAGTGGGTCCGAATGCTTAC
ACCTTCACCGTGGATGTTAATCATGGTCAATGTGGTCACGATGTTCATG
GCGGCTCC

What is claimed is:

1. A recombinant Agaricus bisporus Y3 (Ab-Y3) protein variant comprising an amino acid sequence that is at least 70% identical to the amino acid sequence set forth in SEQ ID NO: 1, and comprises at least one amino acid substitution, insertion, or deletion relative to SEQ ID NO: 1.

2. The recombinant Ab-Y3 protein variant of claim 1, wherein the Ab-Y3 protein variant lacks an N-terminal signal peptide domain (e.g., lacking one or more of amino acids 1-18 of SEQ ID NO: 1).

3. The recombinant Ab-Y3 protein variant of claim 1 or 2, consisting of the amino acid sequence set forth in SEQ ID NO: 3.

4. The recombinant Ab-Y3 protein variant of any one of claims 1 to 3, wherein the recombinant Ab-Y3 protein variant is glycosylated, optionally wherein the glycosylation occurs at a position corresponding to N58 of SEQ ID NO: 1 (e.g., N40 of SEQ ID NO: 3) or wherein the Ab-Y3 protein variant is glycosylated at multiple amino acids.

5. The recombinant Ab-Y3 protein variant of any one of claims 1 to 4, wherein the protein variant binds to one or more types of complex N-glycans.

6. The recombinant Ab-Y3 protein variant of claim 5, wherein the protein variant binds to one or more bi-antennary complex N-glycans.

7. The recombinant Ab-Y3 protein variant of claim 5, wherein the protein variant binds to one or more tri-antennary complex N-glycans.

8. The recombinant Ab-Y3 protein variant of claim 5, wherein the protein variant binds to one or more tetra-antennary complex N-glycans.

9. The recombinant Ab-Y3 protein variant of any one of claims 1 to 8, wherein the protein variant binds to one or more N-glycans shown in FIG. 8.

10. A homo-oligomeric protein complex comprising between 2 and 10 protein subunits, wherein at least one of the subunits consists of the amino acid sequence set forth in SEQ ID NO: 3.

11. An isolated nucleic acid comprising a nucleotide sequence encoding the recombinant Ab-Y3 protein variant of any one of claims 1 to 4, optionally wherein the nucleotide sequence consists of nucleotides 55-408 of SEQ ID NO: 2.

12. An isolated nucleic acid encoding a recombinant Ab-Y3 protein variant, wherein the isolated nucleic acid comprises a nucleotide sequence that is at least 80% identical to the nucleic acid sequence set forth in SEQ ID NO: 2, and comprising at least one nucleotide substitution, deletion, or insertion relative to SEQ ID NO: 2.

13. The isolated nucleic acid of claim 12, wherein the nucleotide sequence comprises nucleotides 55-408 of SEQ ID NO: 2, optionally wherein the nucleotide sequence further comprises a start codon (e.g., ATG) positioned 5′ relative to the nucleotide sequence.

14. The isolated nucleic acid of claim 12, wherein the nucleotide sequence is a codon-optimized nucleotide sequence.

15. The isolated nucleic acid of claim 14, wherein the codon-optimized nucleotide sequence comprises the sequence set forth in SEQ ID NO: 4, optionally wherein the codon-optimized nucleotide sequence further comprises a 5′ start codon (e.g., ATG).

16. The isolated nucleic acid of any one of claims 11 to 15, further comprising a promoter operably linked to the nucleotide sequence encoding the recombinant Ab-Y3 protein variant, optionally wherein the promoter is an inducible AOX1 promoter.

17. A vector comprising the isolated nucleic acid of any one of claims 11 to 16.

18. The vector of claim 17, wherein the vector is a plasmid, optionally a bacterial plasmid vector.

19. A host cell comprising the isolated nucleic acid of any one of claims 11 to 16, or the vector of claim 17 or 18.

20. The host cell of claim 19, wherein the cell is a yeast cell, bacterial cell, or mammalian cell.

21. The host cell of claim 20, wherein the yeast cell is a Pichia cell, optionally a Pichia pastoris cell.

22. A method for determining the presence of a target protein in a sample, the method comprising:
(i) contacting a sample with the recombinant Ab-Y3 protein variant of any one of claims 1 to 9 under conditions sufficient for the recombinant Ab-Y3 protein variant to bind to one or more N-glycans in the sample;
(ii) detecting binding of the recombinant Ab-Y3 protein variant to binding of one or more N-glycans in the sample; and
(iii) determining that a target protein is present in the sample based upon detecting the binding of the recombinant Ab-Y3 protein variant to the one or more N-glycans.

23. The method of claim 22, wherein the target protein is a recombinantly expressed protein, optionally wherein the target protein is expressed in mammalian cells or produced using a cell-free method.

24. The method of claim 22 or 23, wherein the target protein is an immunoglobulin (Ig) protein, optionally wherein the target protein is IgG.

25. A method for isolating an N-glycosylated target protein from a solution, the method comprising:
(i) contacting a solution comprising an N-glycosylated target protein with the Ab-Y3 protein variant of any one of claims 1 to 9 under conditions sufficient for the recombinant Ab-Y3 protein variant to bind to one or more N-glycans of the target protein to form a complex;
(ii) separating the complex from the solution; and
(iii) dissociating the N-glycosylated target protein from the recombinant Ab-Y3 protein variant to form a pure target protein fraction.

26. The method of claim 25, wherein the N-glycosylated target protein is a recombinantly produced protein, optionally wherein the target protein is expressed in mammalian cells.

27. The method of claim 25 or 26, wherein the N-glycosylated target protein is an immunoglobulin (Ig) protein, optionally wherein the target protein is IgG.

28. The method of any one of claims 25 to 27, wherein the recombinant Ab-Y3 protein variant is bound to a solid substrate.

29. The method of claim 28, wherein the solid substrate comprises one or more beads, a gel, or a resin.

30. The method of any one of claims 25 to 29, wherein the separating comprises physically separating the complex from the solution.

31. The method of any one of claims 25 to 30, wherein the dissociating comprises breaking one or more covalent bonds between the recombinant Ab-Y3 protein and the N-glycosylated target protein.

32. The recombinant Ab-Y3 protein variant of any one of claims 1 to 3, wherein the recombinant Ab-Y3 protein variant is unglycosylated.

33. The method of any one of claims 22 to 24, wherein the detecting comprises performing a Western blot or a fluorescence-based N-glycan binding assay.
